(19) United States
(12) Patent Application Publication
(10) Pub. No.: US 2024/0174597 A1
(43) Pub. Date: May 30, 2024

(54) IODINE LABELED HYDROGELS AND CROSSLINKING AGENTS FOR FORMING THE SAME

(71) Applicant: Boston Scientific Scimed, Inc., Maple Grove, MN (US)

(72) Inventor: Yen-Hao Hsu, Shrewsbury, MA (US)

(73) Assignee: Boston Scientific Scimed, Inc., Maple Grove, MN (US)

(21) Appl. No.: 18/514,650

(22) Filed: Nov. 20, 2023

Related U.S. Application Data
(60) Provisional application No. 63/426,867, filed on Nov. 21, 2022.

(51) Int. Cl. C07C 231/14; C08G 64/02

(52) U.S. Cl. CPC C07C 231/14; C08G 64/0233

(57) ABSTRACT

In some embodiments, the present disclosure provides iodinated polyamino compounds having a plurality of primary amine groups, all of which are the same, as well as methods for forming the same. In some embodiments, the present disclosure pertains to systems for forming hydrogels, which systems comprise such an iodinated polyamino compound and a polymer that that forms crosslinks with the iodinated polyamino compound. In some embodiments, the present disclosure pertains to hydrogels that are formed using such systems.

### CROSS-REFERENCE TO RELATED APPLICATIONS

This application claims the benefit of U.S. Provisional Patent Application Ser. No. 63/426,867 filed on Nov. 21, 2022, the disclosure of which is incorporated herein by reference.

### FIELD

The present disclosure relates to iodinated compounds, to hydrogels formed from iodinated compounds, and to methods of making and using iodinated compounds and hydrogels, among other aspects. The iodinated compounds of the present disclosure are useful, for example, in forming hydrogels for various biomedical applications.

### BACKGROUND

In vivo crosslinked hydrogels based on star-poly(ethylene glycol) (star-PEG) polymers functionalized with reactive ester end groups which react with lysine trimer (Lys-Lys-Lys) as a crosslinker to rapidly form crosslinked hydrogels, such as SpaceOAR®, have become clinically significant materials as adjuvants in radiotherapies. See “Augmenix Announces Positive Three-year SpaceOAR® Clinical Trial Results,” Imaging Technology News, Oct. 27, 2016.

Hydrogels in which some of the star-PEG branches have been functionalized with 2,3,5-triiodobenzamide (TIB) groups replacing part of ester end groups, such SpaceOAR® Vue, have also been developed to provide enhanced radiocontrast properties. See “Augmenix Receives FDA Clearance to Market its TraceIT® Tissue Marker,” BusinessWire Jan. 28, 2013. TraceIT® hydrogel remains stable and visible in tissue for a minimum of three months, long enough for radiotherapy, after which it is absorbed and cleared from the body.

While the above approach is effectual, using some of the arms of the star polymer to functionalize the hydrogel with iodine means that there are fewer arms available to crosslink. This can be overcome by adding more polymer, but the loading of solids increases, which can adversely impact viscosity. Reducing the molecular weight can cut down on the loading of solids, but this also results in a lower melting point, and problems with processability. An additional effect of the reduced crosslink density per star polymer is that the resulting gel has a slower cure rate, which means the gel is liquid and mobile in vivo for longer time periods, opening up opportunities for unintended side-reactions and material displacement. Moreover, TIB is sparingly water soluble, meaning that there is an upper limit to how much iodine can be added before the solubility of the gel becomes impacted. In the event that the concentration of TIB groups is so high that the star-PEG precipitates out of solution, the TIB groups can physically crosslink the system before it even reacts, requiring greater force to dispense. Finally, star PEG labeled with 2,3,5-triiodobenzamide end groups often show discoloration from thermal degradation. While this doesn't impact their functionality, this is a cosmetic defect that is preferably avoided.

There is thus a continuing need in the biomedical arts for additional hydrogels, for precursors of such hydrogels, for methods of making such hydrogels and precursors, for methods of using such hydrogels and precursors, and for systems for forming such hydrogels, among other needs.

### SUMMARY

The present disclosure provides an alternative approach to that described above. Rather than using the arms of the polymer to functionalize the hydrogel with iodine, in the present disclosure, the crosslinker for the polymer is functionalized with iodine.

In various embodiments, the present disclosure provides iodinated polyamino compounds having a plurality of primary amine groups, all of which are the same.

In some embodiments, the plurality of primary amine groups are alkylamino groups. In some of these embodiments, the alkylamino groups are of the formula —(CH₂)ₓ—NH₂, where x is 1, 2 3, 4, 5 or 6 and/or all of the alkylamino groups are attached to the same carbon atom.

In some embodiments, the iodinated polyamino compound comprises a polyamino moiety comprising the plurality of primary amine groups that is linked to an iodinated moiety by an amide group.

In some embodiments, the polyamino moiety is a —(CH₂)ₓC((CH₂)ₓ—NH₂)₃ moiety, where x is 1, 2 3, 4, 5 or 6.

In some embodiments, which are applicable to any of the preceding embodiments, the iodinated moiety comprises an iodinated aromatic group.

In some embodiments, which are applicable to any of the preceding embodiments, the iodinated moiety comprises a monocyclic or multicyclic aromatic moiety that is substituted with one or more iodine groups and one or more hydroxyl-containing groups.

In accordance with further embodiments, the present disclosure provides systems for forming hydrogels, which comprise an iodinated polyamino compound in accordance with any of the above embodiments and a polymer that that forms crosslinks with the iodinated polyamino compound.

In some embodiments, the polymer that that forms crosslinks with the iodinated polyamino compound is reactive multi-arm polymer that comprises a plurality of hydrophilic polymer arms having reactive end groups that covalently crosslink with the primary amine groups of the iodinated polyamino compound. In some of these embodiments, the hydrophilic polymer arms comprise one or more hydrophilic monomers selected from ethylene oxide, N-vinyl pyrrolidone, oxazolines, hydroxyethyl acrylate, hydroxyethyl methacrylate, PEG methyl ether acrylate or PEG methyl ether methacrylate, or PNIPAAM and/or the reactive end groups are linked to the hydrophilic polymer arms by a hydrolysable ester and/or the reactive end groups are electrophilic groups, which may be selected, for example, from imidazole esters, imidazole carboxylates, benzotriazole esters, or imide esters.

In some embodiments, the polymer that forms crosslinks with the iodinated polyamino compound is an anionic polymer that comprises a plurality of anionic groups that can ionically crosslink with the primary amine groups of the iodinated polyamino compound. For example, the anionic polymer may comprise a plurality of carboxyl groups.

In some embodiments, which are applicable to any of the preceding embodiments, the systems comprise a first precursor composition that comprises the iodinated polyamino compound, a second precursor composition that comprises the polymer that forms crosslinks with the iodinated polyamino compound, and an optional accelerant composition. For example, the first precursor composition may be provided in a syringe barrel, the second precursor composition may be provided in a vial, and the accelerant composition may be provided in a syringe barrel.

In some embodiments, which are applicable to any of the preceding embodiments, the systems may further comprise a delivery device.

In some embodiments, the present disclosure provides medical hydrogels that are formed by crosslinking an iodinated polyamino compound in accordance with any of the preceding embodiments and a polymer that forms crosslinks with the iodinated polyamino compound in accordance with any of the preceding embodiments.

In some embodiments, the present disclosure provides methods of treatment comprising administering to a subject a mixture that comprises an iodinated polyamino compound in accordance with any of the preceding embodiments and a polymer that forms crosslinks with the iodinated polyamino compound in accordance with any of the preceding embodiments, under conditions such that the iodinated polyamino compound and the polymer crosslink after administration.

In some embodiments, the present disclosure provides methods of forming an iodinated polyamino compound that comprise (a) forming a partially protected polyamino compound by protecting all but one primary amine group of a polyamino precursor compound comprising three or more primary amine groups, (b) forming an amide linkage between a remaining unprotected primary amine group of the partially protected polyamino compound and a carboxyl group of a carboxylated iodinated precursor compound, and (c) deprotecting primary groups of the product of step (b).

Potential benefits associated with the present disclosure include one or more of the following: radiocontrast is maintained, complexity and cost of the manufacturing process is reduced, melting point of the solid components of the hydrogel can be maintained above 40° C. (improving storage and handling), homogeneity of the final hydrogel is improved, in vivo persistence is obtained, and cure kinetics are maintained. Moreover, because the iodinated crosslinker contains a plurality of primary amine groups, all of which are the same, the primary amine groups have the same reactivity, providing the potential to efficiently and consistently form radiopaque crosslinked gels.

The above and other aspects, embodiments, features and benefits of the present disclosure will be readily apparent from the following detailed description.

### BRIEF DESCRIPTION OF THE DRAWINGS

FIG. 1 illustrates a method of forming an iodinated polyamino compound, in accordance with an embodiment of the present disclosure.

FIG. 2 schematically illustrates a method whereby a polymer, which comprises a core region and a plurality of hydrophilic polymer arms having reactive end groups, is crosslinked with an iodinated polyamino compound, according to an aspect of the present disclosure.

FIG. 3 schematically illustrates a method whereby a hyaluronic acid polymer is crosslinked with an iodinated polyamino compound, according to an aspect of the present disclosure.

### DETAILED DESCRIPTION

In some aspects of the present disclosure, a radiopaque crosslinked hydrogel is provided that comprises a crosslinked reaction product of (a) an iodinated polyamino compound and (b) a polymer that forms crosslinks with the iodinated polyamino compound. Unless indicated otherwise, as used herein the prefix “poly” means two or more.

In various embodiments, the iodinated polyamino compounds of the present disclosure comprise a polyamino moiety that is linked to an iodinated moiety through an amide group.

In some embodiments, polyamino moiety has a plurality (two, three, four, five, six, seven, eight, nine, ten or more) of primary amine groups, all of which are the same. For example, the polyamino moiety may comprises a plurality of (two, three, four, five, six, seven, eight, nine, ten or more) identical —(CH₂)ₓ—NH₂ groups where x is 1, 2 3, 4, 5 or 6. In some of these embodiments, three identical —(CH₂)ₓ—NH₂ groups are attached to a single carbon atom core. Specific examples of polyamino moieties include —(CH₂)ₓC((CH₂)ₓ—NH₂)₃ moieties where x is 1, 2 3, 4, 5 or 6, for example, a —CH₂C(CH₂NH₂)₃ moiety,

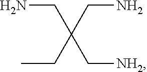

a —CH₂CH₂C(CH₂CH₂NH₂)₃ moiety,

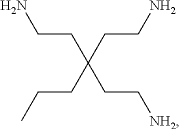

and a —CH₂CH₂CH₂C(CH₂CH₂CH₂NH₂)₃ moiety,

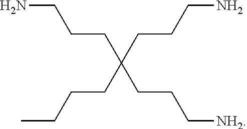 [SMILES: CCCCC(CCCN)(CCCN)CCCN]

As previously indicated, in addition to a polyamino moiety, such as one of those described above, the iodinated polyamino compounds of the present disclosure also comprise an iodinated moiety linked to the polyamino moiety. The iodinated moiety is linked to the polyamino moiety by an amide linkage.

In some embodiments, the iodinated moiety comprises an iodinated aromatic group (also referred to as an iodo-aromatic group). Examples of iodinated aromatic groups include iodine-substituted monocyclic aromatic groups and iodine-substituted multicyclic aromatic groups, such as iodo-phenyl groups and iodo-naphthyl groups. The aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.).

Examples of iodinated aromatic groups include those that comprise one or more monocyclic or multicyclic aromatic structures, such as a benzene or naphthalene structure, that is substituted with (a) one or more iodine groups (e.g., one two, three, four, five, six or more iodine atoms) and (b) one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from amide groups, amine groups, ether groups, ester groups, or carbonate groups, among others.

Examples of iodinated aromatic groups include those that comprise at least one hydroxy-iodo-aromatic group and/or at least one C₁-C₄-hydroxyalkyl-iodo-aromatic group. Examples of hydroxy-iodo-aromatic groups include hydroxy-iodo-phenyl groups, C₁-C₄-hydroxyalkyl-iodo-phenyl groups, hydroxy-iodo-naphthyl groups, and C₁-C₄-hydroxyalkyl-iodo-naphthyl groups. More particular examples of hydroxy-iodo-aromatic groups include (a) hydroxy-iodo-phenyl groups selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a tri-hydroxy-mono-iodo-phenyl group, and a tri-hydroxy-di-iodo-phenyl group and (b) C₁-C₄-hydroxyalkyl-iodo-aromatic groups include C₁-C₄-hydroxyalkyl-iodo-phenyl groups selected from a mono-C₁-C₄-hydroxyalkyl-mono-iodo-phenyl group, a mono-C₁-C₄-hydroxyalkyl-di-iodo-phenyl group, a mono-C₁-C₄-hydroxyalkyl-tri-iodo-phenyl group, a mono-C₁-C₄-hydroxyalkyl-tetra-iodo-phenyl group, a di-C₁-C₄-hydroxyalkyl-mono-iodo-phenyl group, a di-C₁-C₄-hydroxyalkyl-di-iodo-phenyl group, a di-C₁-C₄-hydroxyalkyl-tri-iodo-phenyl group, a tri-C₁-C₄-hydroxyalkyl-mono-iodo-phenyl group, and a tri-C₁-C₄-hydroxyalkyl-di-iodo-phenyl group.

Specific examples of iodinated moieties include the following, among many others:

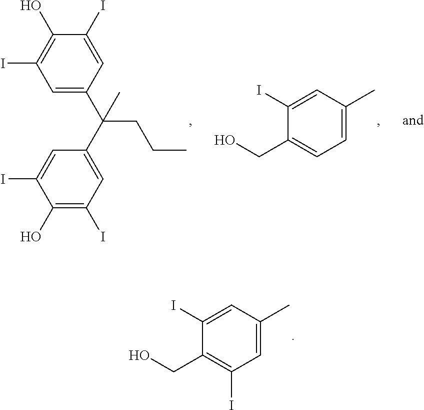

The amide group linking the polyamino moiety with the iodinated moiety may be formed in an amide coupling reaction between a carboxyl group of a carboxylated iodinated precursor compound and a primary amine group of a polyamino precursor compound. The resulting iodinated polyamino compounds therefore comprise a residue of the polyamino precursor compound and a residue of the carboxylated iodinated precursor compound.

Examples of carboxylated iodinated precursor compounds include those that comprise one or more iodine groups, one or more carboxyl groups and one or more hydroxyl-containing groups. In various embodiments, the carboxylated iodinated precursor compounds may comprise a monocyclic or multicyclic aromatic structure, such as a benzene or naphthalene structure, that is substituted with (a) one or more iodine groups (e.g., one two, three, four, five, six or more iodine atoms), (b) one or more carboxyl-containing groups such as carboxyl groups and/or C₂-C₆-carboxyalkyl (e.g., carboxymethyl, carboxyethyl, carboxypropyl, carboxybutyl, etc.) groups, among others, which carboxyalkyl groups may be linked to the monocyclic or multicyclic aromatic structures directly or through any suitable linking moiety, which may be selected, for example, from amide groups, amine groups, ether groups, ester groups, or carbonate groups, among others, and (c) one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from amide groups, amine groups, ether groups, ester groups, or carbonate groups, among others. Specific examples of carboxylated iodinated precursor compounds include the following, among many others: hydroxymethyl iodobenzoic acid,

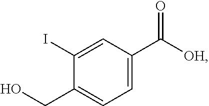 [SMILES: O=C(O)c1ccc(CO)c(I)c1]

hydroxymethyl diiodobenzoic acid,

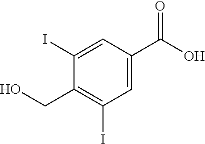 [SMILES: O=C(O)c1cc(I)c(CO)c(I)c1]

and 4,4-bis(hydroxy-3,5-diiodophenyl) pentanoic acid (IBHP),

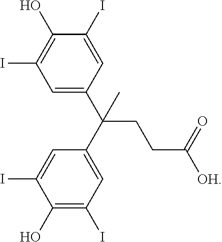 [SMILES: CC(CCC(=O)O)(c1cc(I)c(O)c(I)c1)c1cc(I)c(O)c(I)c1]

Examples of polyamino precursor compounds include compounds having three or more (three, four, five, six, seven, eight, nine, ten or more) identical primary amine groups. For example, the polyamino precursor compounds may comprise three or more (three, four, five, six, seven, eight, nine, ten or more) identical —(CH₂)ₓ—NH₂ groups where x is 1, 2 3, 4, 5 or 6. In some of these embodiments, four identical —(CH₂)ₓ—NH₂ groups are attached to single carbon atom core. Specific examples include compounds of the formula C(—(CH₂)ₓ—NH₂)₄, where x is 1, 2 3, 4, 5 or 6, including C(CH₂NH₂)₄,

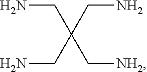

C(CH₂CH₂NH₂)₄,

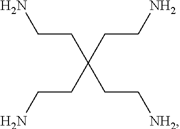 [SMILES: NCCC(CCN)(CCN)CCN]

and C(CH₂CH₂CH₂NH₂)₄,

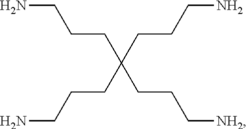 [SMILES: NCCCC(CCCN)(CCCN)CCCN]

among others.

In some aspects, the present disclosure pertains to processes of making iodinated polyamino compounds such as those described above.

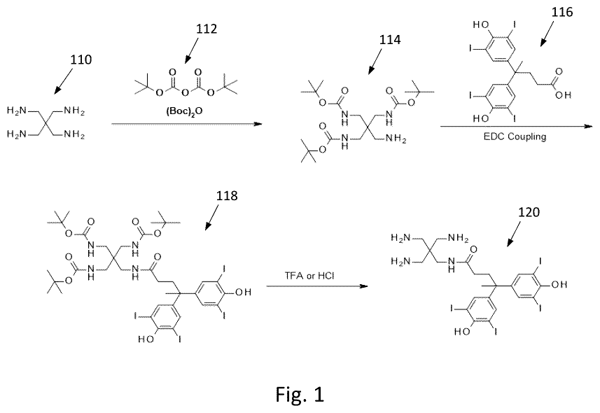

In a first step, all but one of the primary amine groups of a polyamino precursor compound may be protected with a suitable protective agent. The primary amine groups are protected for compatibility with other reactants in a subsequent amide coupling reaction. For example, all but one of the primary amine groups of a polyamino precursor compound may be protected by controlled reaction with di-tert-butyl dicarbonate. In a particular example, and with reference to FIG. 1, all but one of the primary amine groups of 2,2-bis(aminomethyl)propane-1,3-diamine (110) are protected using di-tert-butyl dicarbonate (CAS #24424-99-5) (112), thereby forming partially t-Boc-protected 2,2-bis(aminomethyl)propane-1,3-diamine (114). This leaves the remaining unprotected primary amine groups of the partially protected compound available for amide coupling.

In a second step, a carboxylated iodinated precursor compound is coupled with the partially protected polyamino compound formed in the first process step in an amide coupling reaction (e.g., via a carbodiimide coupling reagent) to form a protected iodinated polyamino compound. In a particular example, and with reference to FIG. 1, the partially t-Boc-protected 2,2-bis(aminomethyl)propane-1,3-diamine (114) from the first step is coupled to a carboxylated iodinated precursor compound (116) (specifically 4,4-bis(hydroxy-3,5-diiodophenyl) pentanoic acid (IBHP)), for example, in the presence of 1-ethyl-3-(3-dimethylaminopropyl)carbodiimide hydrochloride (EDC·HCl) in dimethylformamide (DMF), or another suitable organic solvent such as dimethyl sulfoxide (DMSO), dichloromethane (DCM) or acetonitrile, yielding a t-Boc protected iodinated polyamino compound (118).

In a third step, deprotection of the product of the second step is performed (e.g., under acidic conditions), to form a final iodinated polyamino compound. For example, as shown in FIG. 1, the t-Boc protected iodinated polyamino compound (118) is deprotected under acidic conditions using an acid such as trifluoroacetic acid or hydrochloric acid to form an iodinated polyamino compound (120).

The process described above can be performed using a variety of polyamino precursor compounds and a variety of carboxylated iodinated precursor compounds.

As noted above, in various aspects of the present disclosure, a radiopaque crosslinked hydrogel is provided that comprises a crosslinked reaction product of (a) an iodinated polyamino compound such one of those described above and (b) a polymer that forms crosslinks with the iodinated polyamino compound.

In various embodiments, the crosslinked products of the present disclosure are visible under fluoroscopy. In various embodiments, such crosslinked products have a radiopacity that is 250 Hounsfield units (HU) or more, beneficially anywhere ranging from 250 HU to 500 HU to 750 HU to 1000 HU to 2500 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In some embodiments, the polymer that forms crosslinks with the iodinated polyamino compound is a reactive polymer that comprises a plurality of reactive groups that form covalent crosslinks with the primary amine groups of the iodinated polyamino compound.

Examples of reactive polymers that comprise a plurality of reactive groups that form covalent crosslinks with the primary amine groups of the iodinated polyamino compound include multi-arm polymers that comprise a plurality of polymer arms (e.g., having two, three, four, five, six, seven, eight, nine, ten, eleven, twelve or more arms), wherein two or more polymer arms of the multi-arm polymer comprise one or more reactive end groups. In some embodiments, compositions containing the reactive multi-arm polymers may be provided in which a percentage of the polymer arms comprising one or more reactive end groups may correspond to between 50% and 100% of the total number of polymer arms in the composition (e.g., ranging anywhere from 50% to 70% to 80% to 90% to 95% to 99% to 100% of the total number of polymer arms). Typical average molecular weights for the reactive multi-arm polymers for use herein are of at least 10 kDa, in some cases ranging from 10 kDa to 50 kDa or more. In various embodiments, the reactive multi-arm polymers for use herein have a melting point of 40° ° C.for greater, preferably 45° C.for greater.

In various embodiments, the polymer arms of the multi-arm polymers are hydrophilic polymer arms. Such hydrophilic polymer arms may be composed of any of a variety of synthetic, natural, or hybrid synthetic-natural polymers including, for example, poly(alkylene oxides) such as poly(ethylene oxide) (PEO, also referred to as polyethylene glycol or PEG), poly(propylene oxide) or poly(ethylene oxide-co-propylene oxide), poly(N-vinyl pyrrolidone), polyoxazolines including poly(2-alkyl-2-oxazolines) such as poly(2-methyl-2-oxazoline), poly(2-ethyl-2-oxazoline) and poly(2-propyl-2-oxazoline), poly(vinyl alcohol), poly(allyl alcohol), polyhydroxyethyl acrylate, polyhydroxyethyl methacrylate, PEG methyl ether acrylate or PEG methyl ether methacrylate, or PNIPAAM, polysaccharides, and combinations thereof. Such hydrophilic polymer arms may comprise one or more hydrophilic monomers selected from ethylene oxide, N-vinyl pyrrolidone, a 2-alkyl-2-oxazoline monomer, hydroxyethyl acrylate, hydroxyethyl methacrylate, PEG methyl ether acrylate or PEG methyl ether methacrylate, or PNIPAAM.

In some embodiments, the polymer arms of the reactive multi-arm polymers extend from a core region. In certain of these embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, tripentaerythritol, adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others. In certain beneficial embodiments, the core region comprises a residue of a polyol that contains two, three, four, five, six, seven, eight, nine, ten or more hydroxyl groups.

In certain of these embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, when the core region comprises a silsesquioxane, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

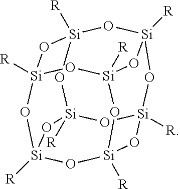

In the present disclosure, when the core region comprises a silsesquioxane, at least two R groups comprise polymer arms, and typically all R groups comprise polymer arms.

In certain embodiments, the reactive end groups may be electrophilic groups selected from imidazole esters, imidazole carboxylates, benzotriazole esters, or imide esters, including N-hydroxysuccinimidyl esters. A particularly beneficial reactive end group is an N-hydroxysuccinimidyl ester group. In certain embodiments, the reactive end groups are linked to the polymer arms via hydrolysable ester groups. Hydrolysable ester groups may be selected, for example, from glutarate ester groups, succinate ester groups, carbonate ester groups, or adipate ester groups. In particular embodiments, the polymer arms may be terminated with the following reactive, hydrolysable groups, among others: succinimidyl glutarate groups, succinimidyl succinate groups, succinimidyl carbonate groups, or succinimidyl adipate groups.

Further examples of reactive multi-arm polymers are described, for example, in U.S. Patent Application Nos. 2011/0142936, 2021/0061950, 2021/0061954 and 2021/0061957.

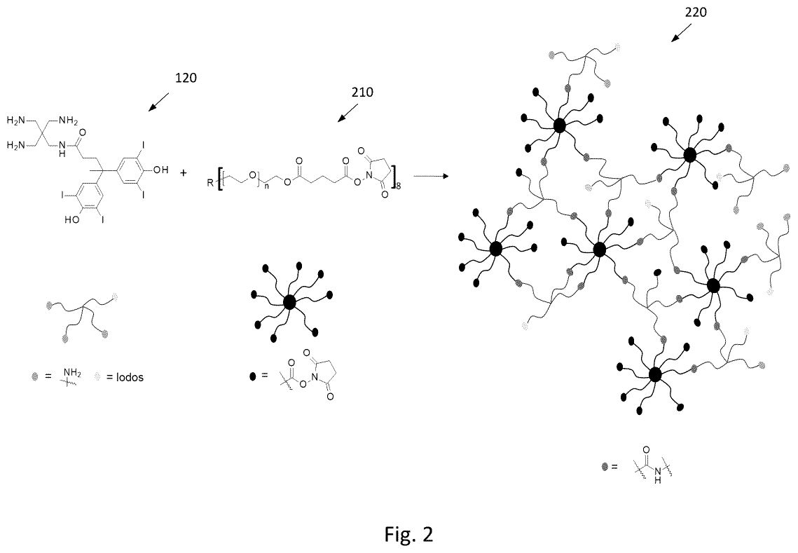

In a particular example shown schematically in FIG. 2, a reactive multi-arm polymer (210) like that described above, which comprises a core region and a plurality of hydrophilic polyethylene oxide arms, specifically eight arms) having reactive end groups (i.e., succinimidyl glutarate groups) (where R is a core region, such as a polyol residue (e.g., a pentaerythritol residue), and n ranges, for example, from 25 to 140) can be covalently crosslinked with an iodinated polyamino compound (120) like that described above, which comprises primary amine groups that are reactive with the reactive groups (i.e., succinimidyl glutarate groups) of the reactive multi-arm polymer (210). By reacting the iodinated polyamino compound (120) with the reactive multi-arm polymer (210) under basic conditions, a crosslinked product (220) is formed, which may be in the form of a crosslinked hydrogel when hydrated.

An advantage to this approach is that iodine functionality, and thus radiopacity, is provided by the iodinated polyamino compound that acts as a crosslinker for the reactive multi-arm polymer. This allows reactive end groups to be provided on each of the polymer arms, thereby maximizing the crosslinking capacity of the reactive multi-arm polymer, without sacrificing radiopacity. Moreover, since the iodination is separate from the reactive polymer, and the multi-arm polymer can be swapped out with N-hydroxysuccinimidyl-ester-functionalized systems having hydrophilic polymer arms other than polyethylene oxide arms, for example, synthetic, natural, or hybrid synthetic-natural hydrophilic polymer arms such as those described above.

Other advantages to this approach stem from the fact that the primary amine groups (typically C₁-C₆ alkylamino groups) of the iodinated polyamino compound are the same. One effect of this is that the primary amine groups have the same reactivity, thereby providing the potential to efficiently and consistently form radiopaque crosslinked gels. This is in contrast to other known crosslinkers such as trilysine,

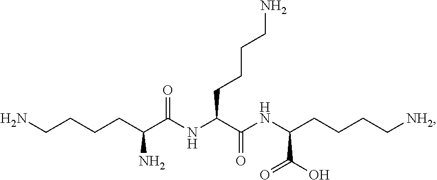 [SMILES: NCCCC[C@H](NC(=O)[C@H](CCCCN)NC(=O)[C@@H](N)CCCCN)C(=O)O]

which has four primary amine groups, one of which has more steric hindrance than the others, which could potentially cause incomplete curing or slow the curing rate.

As previously noted, in various aspects of the present disclosure, a radiopaque crosslinked hydrogel is provided that comprises a crosslinked reaction product of (a) an iodinated polyamino compound such as those described above and (b) a polymer that forms crosslinks with the iodinated polyamino compound.

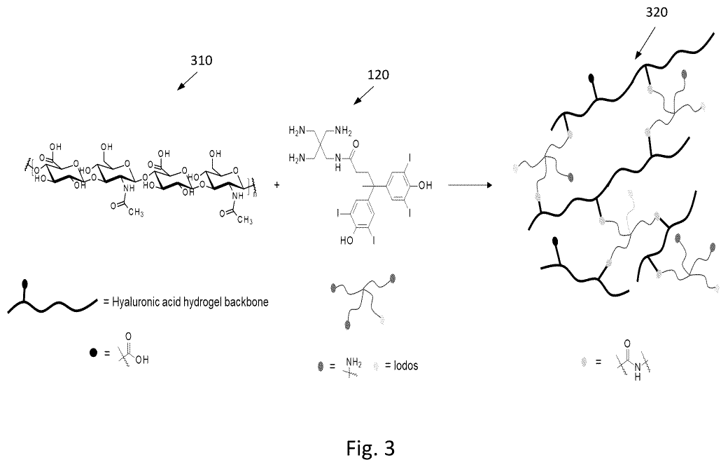

In some embodiments, the polymer that forms crosslinks with the iodinated polyamino compound may be a poly(carboxylic acid), such as poly(acrylic acid), poly(methacrylic acid), alginic acid, hyaluronic acid, pectin, agaropectin, carrageenan, gellan gum, gum arabic, guar gum, xanthan gum, and carboxymethyl cellulose. These and other poly(carboxylic acids) have carboxylic acid groups that form amide bonds with primary amine groups of the iodinated polyamino compound. Such crosslinks may be formed, for example, in the presence of a carbodiimide coupling reagent as previously described. In a particular example shown schematically in FIG. 3, hyaluronic acid (310) is covalently crosslinked with an iodinated polyamino compound (120) like that described above, which comprises primary amine groups that form amide bonds with the carboxylic acid groups of the hyaluronic acid (310), thereby forming a crosslinked product (320), which may be in the form of a crosslinked hydrogel when hydrated.

In some embodiments, the polymer that forms crosslinks with the iodinated polyamino compound is an anionic polymer that comprises a plurality of anionic groups that can ionically crosslink with cationic primary amine groups of the iodinated polyamino compound. In some embodiments, the anionic polymer is water soluble.

Examples of anionic polymers that comprise a plurality of anionic groups that can ionically crosslink with cationic primary amine groups of the iodinated polyamino compound include any of a variety of synthetic, natural, or hybrid synthetic-natural anionic polymers that comprise one or more groups selected from carboxylate groups, sulfonate groups, sulfate groups, phosphate groups, or phosphonate groups, which are negatively charged at a pH greater than or equal to 7 or more and in some cases are negatively charged at a pH greater than 6, greater than 4, or even greater than 2. Particular examples of anionic polymers include poly(carboxylic acids) such as poly(acrylic acid) or poly(methacrylic acid), anionic polysaccharides including alginates, hyaluronates, pectins, agaropectins, carrageenans, gellan gum, gum arabic, guar gum, xanthan gum, and carboxymethyl celluloses, sulfonate polymers such as poly(2-acrylamido-2-methylpropane sulfonate) (polyAMPS) or polystyrene sulfonate, and polyphosphates. The anionic polymer may be provided in a salt form, for example, in a sodium salt form or a potassium salt form, among others.

An advantage to this approach is that iodine functionality, and thus radiopacity, is provided by the iodinated polyamino compound that acts as a cationic crosslinker for the anionic polymer. Because the iodination is separate from the anionic polymer, the anionic polymer can be swapped out with other anionic polymers, for example, synthetic, natural, or hybrid synthetic-natural anionic polymers such as those described above.

In some aspects of the present disclosure, systems are provided that are configured to dispense an iodinated polyamino compound and a polymer that forms crosslinks with the iodinated polyamino compound under conditions such that the iodinated polyamino compound and the polymer crosslink with one another. Such systems can be used to form crosslinked hydrogels, either in vivo or ex vivo.

In some embodiments, systems are provided that are configured to dispense an iodinated polyamino compound and a reactive polymer that comprises a plurality of reactive groups that form covalent crosslinks with the primary amine groups of the iodinated polyamino under conditions such that the iodinated polyamino compound and the reactive polymer covalently crosslink with one another. Exemplary iodinated polyamino compounds and reactive polymers are described above and include reactive multi-arm polymers that comprise a plurality of polymer arms, wherein two or more polymer arms of the multi-arm polymer comprise one or more reactive end groups. In certain embodiments, those conditions comprise an environment having a basic pH, for example, a pH ranging from about 9 to about 11, typically ranging from about 9.5 to about 10.5, and beneficially ranging from about 9.8 to about 10.2.

In some embodiments, a system is provided that comprises (a) a first composition that comprises an iodinated polyamino compound and (b) a second composition that comprises a reactive polymer that forms covalent crosslinks with the iodinated polyamino compound. In some embodiments, a third composition in the form of an accelerant composition is provided.

The first composition may be a first fluid composition comprising the iodinated polyamino compound or a first dry composition that comprises the iodinated polyamino compound, to which a suitable fluid such as water for injection, saline, etc. can be added to form a first fluid composition. In addition to the iodinated polyamino compound, the first composition may further comprise additional agents, including those described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents including those described below.

In some embodiments, the iodinated polyamino compound is initially combined with the reactive multi-arm polymer at an acidic pH at which covalent crosslinking between the reactive groups of the reactive multi-arm polymer and the primary amine groups of the iodinated polyamino compound is suppressed. Then, when covalent crosslinking is desired, a pH of the mixture of the iodinated polyamino compound and the reactive multi-arm polymer is changed from an acidic pH to a basic pH, leading to covalent crosslinking between same.

In particular embodiments, the system comprises (a) a first precursor composition that comprises an iodinated polyamino compound as described hereinabove, (b) a second precursor composition that comprises a reactive multi-arm polymer as described hereinabove, and (c) a third composition, specifically, an accelerant composition, that contains an accelerant that is configured to accelerate crosslinking reaction between the iodinated polyamino compound and the reactive multi-arm polymer.

The first precursor composition may be a first fluid composition comprising the iodinated polyamino compound that is buffered to an acidic pH or a first dry composition that comprises the iodinated polyamino compound and acidic buffering composition, to which a suitable fluid such as water for injection, saline, etc. can be added to form a first fluid composition comprising the iodinated polyamino compound that is buffered to an acidic pH. In some embodiments, for example, the acidic buffering composition may comprise monobasic sodium phosphate, among other possibilities. The first fluid composition comprising the iodinated polyamino compound may have a pH ranging, for example, from about 3 to about 5, typically ranging from about 3.5 to about 4.5, and more typically ranging from about 3.8 to about 4.2. In addition to the iodinated polyamino compound, the first precursor composition may further comprise additional agents, including those described below.

The second precursor composition may be a second fluid composition comprising the reactive multi-arm polymer or a second dry composition that comprises the reactive multi-arm polymer from which a second fluid composition is formed, for example, by the addition of a suitable fluid such as water for injection, saline, or by the addition of the first fluid composition comprising the iodinated polyamino compound that is buffered to an acidic pH. In addition to the reactive multi-arm polymer, the second precursor composition may further comprise additional agents, including those described below.

In a particularly beneficial embodiment, the first precursor composition is a first fluid composition comprising the iodinated polyamino compound that is buffered to an acidic pH and the second precursor composition comprises a dry composition that comprises the reactive multi-arm polymer. The first precursor composition may then be mixed with the second precursor composition to provide a prepared fluid composition that is buffered to an acidic pH and comprises the iodinated polyamino compound and the reactive multi-arm polymer. In a particular example, a syringe may be provided that contains a first fluid composition comprising the iodinated polyamino compound that is buffered to an acidic pH, and a vial may be provided that comprises a dry composition (e.g., a powder) that comprises the reactive multi-arm polymer. The syringe may then be used to inject the first fluid composition into the vial containing the reactive multi-arm polymer to form a prepared fluid composition that contains the iodinated polyamino compound and the reactive multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11, typically ranging from about 9.5 to about 10.5, and more typically ranging from about 9.8 to about 10.2. In addition to the above, the fluid accelerant composition may further comprise additional agents, including those described below. In a particular example, a syringe may be provided that contains the fluid accelerant.

A prepared fluid composition that is buffered to an acidic pH and comprises the iodinated polyamino compound and the reactive multi-arm polymer as described above, and a fluid accelerant composition that is buffered to basic pH as described above, may be combined form crosslinked hydrogels, either in vivo or ex vivo.

In some embodiments, systems are provided that are configured to dispense an iodinated polyamino compound and an anionic polymer that comprises a plurality of anionic groups that ionically crosslink with cationic primary amine groups of the iodinated polyamino compound under pH conditions such that the iodinated polyamino compound and the anionic polymer ionically crosslink with one another. Exemplary iodinated polyamino compounds and anionic polymers are described above.

In some embodiments, a system is provided that comprises (a) a first composition that comprises an iodinated polyamino compound and (b) a second composition that comprises an anionic polymer that forms ionic crosslinks with the iodinated polyamino compound. In some embodiments, a third composition in the form of an accelerant composition is provided.

The first composition may be a first fluid composition comprising the iodinated polyamino compound or a first dry composition that comprises the iodinated polyamino compound, to which a suitable fluid such as water for injection, saline, etc. can be added to form a first fluid composition. In addition to the iodinated polyamino compound, the first composition may further comprise additional agents, including those described below.

The second composition may be a second fluid composition comprising the anionic polymer or a second dry composition that comprises the anionic polymer, to which a suitable fluid such as water for injection, saline, etc. can be added to form a second fluid composition. In addition to the anionic polymer, the second composition may further comprise additional agents, including those described below.

In some embodiments, the first composition may be a first fluid composition comprising the iodinated polyamino compound that is buffered to acidic pH (e.g., ranging from 3.8 to 4.2) a first dry composition that comprises the iodinated polyamino compound and a buffering composition, to which a suitable fluid such as water for injection, saline, etc. can be added to form a first fluid composition comprising the iodinated polyamino compound that is buffered to acidic pH. At acidic pH the iodinated polyamino compound is positively charged, having enhanced water solubility.

In some embodiments, the second composition may be second fluid composition comprising the anionic polymer that is buffered to basic pH or a second dry composition that comprises the anionic polymer and a buffering composition, to which a suitable fluid such as water for injection, saline, etc. can be added to form a second fluid composition comprising the anionic polymer that is buffered to basic pH. Where the anionic polymer comprises carboxyl groups, at basic pH the carboxyl groups are negatively charged, having enhanced water solubility.

The buffering agents in the first and second fluid compositions are selected such that when the first and second fluid compositions are combined, the pH of the resulting mixture is such that the primary amine groups of the iodinated polyamino compound are positively charged and carboxyl groups of the iodinated polyamino compound are negatively charged, such that an ionically crosslinked hydrogel is formed.

A first fluid composition comprising the iodinated polyamino compound that is buffered to acidic pH may be combined, either in vivo or ex vivo, with a second fluid composition comprising the anionic polymer that is buffered to basic pH to form a crosslinked hydrogel.

In some embodiments, the iodinated polyamino compound is initially combined with the anionic polymer either at a basic pH at which the primary amine groups of the iodinated polyamino compound are neutrally charged, or at an acidic pH at which the carboxyl groups of the anionic are neutrally charged, such that ionic crosslinking is suppressed. Then, when crosslinking is desired, a pH of the mixture of the iodinated polyamino compound and the anionic polymer is changed from an acidic or basic pH to a more neutral pH, leading to ionic crosslinking between the iodinated polyamino compound and the anionic polymer.

In particular embodiments, the system comprises (a) a first precursor composition that comprises an iodinated polyamino compound as described hereinabove, (b) a second precursor composition that comprises an anionic polymer as described hereinabove, and (c) a third composition, specifically, an accelerant composition, that contains an accelerant that is configured to accelerate crosslinking reaction between the iodinated polyamino compound and the anionic polymer.

The first precursor composition may be a first fluid composition comprising the iodinated polyamino compound that is buffered to an acidic or basic pH or a first dry composition that comprises the iodinated polyamino compound and an acidic or basic buffering composition, to which a suitable fluid such as water for injection, saline, etc. can be added to form a first fluid composition comprising the iodinated polyamino compound that is buffered to an acidic or basic pH. In addition to the iodinated polyamino compound, the first precursor composition may further comprise additional agents, such as those descried below.

The second precursor composition may be a second fluid composition comprising the anionic polymer or a second dry composition that comprises the anionic polymer from which a fluid composition is formed, for example, by the addition of a suitable fluid such as water for injection, saline, or the first fluid composition comprising the iodinated polyamino compound that is buffered to an acidic or basic pH. In addition to the anionic polymer, the second precursor composition may further comprise additional agents, such as those descried below.

In a particularly beneficial embodiment, the first precursor composition is a first fluid composition comprising the iodinated polyamino compound that is buffered to an acidic or basic pH and the second precursor composition comprises a dry composition that comprises the anionic polymer. The first precursor composition may then be mixed with the second precursor composition to provide a prepared fluid composition that is buffered to an acidic or basic pH and comprises the iodinated polyamino compound and the anionic polymer. In a particular example, a syringe may be provided that contains a first fluid composition comprising the iodinated polyamino compound that is buffered to an acidic or basic pH, and a vial may be provided that comprises a dry composition (e.g., a powder) that comprises the anionic polymer. The syringe may then be used to inject the first fluid composition into the vial containing the anionic polymer to form a prepared fluid composition that contains the iodinated polyamino compound and the anionic polymer, which can be withdrawn back into the syringe for administration.

The accelerant composition may be a fluid accelerant composition that is buffered to a basic or acidic pH or a dry composition that comprise a basic buffering composition to which a suitable fluid such as water for injection, saline, etc. can be added to form a fluid accelerant composition that is buffered to a basic or acidic pH. The amount and type of buffering agent in the fluid accelerant composition are selected such that when combined with the prepared fluid composition that contains the iodinated polyamino compound and the anionic polymer, the resulting mixture has a more neutral pH at which the that iodinated polyamino compound is positively charged and the anionic polymer is negatively charged, such that ionic crosslinks form between the same. In addition to the above, the fluid accelerant composition may further comprise additional agents, such as those descried below.

A prepared fluid composition that is buffered to an acidic or basic pH and comprises the iodinated polyamino compound and the anionic polymer as described above, and a fluid accelerant composition that is buffered to a basic or acidic pH as described above, may be combined form crosslinked hydrogels, either in vivo or ex vivo.

Examples of additional agents for use in the above-described compositions include therapeutic agents such anti-angiogenic agents, cytotoxic agents, chemotherapeutic agents, checkpoint inhibitors, immune modulatory cytokines, T-cell agonists, and STING (stimulator of interferon genes) agonists.

Examples of additional agents further include imaging agents in addition to the iodine present in the radiopaque products. Such imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ⁹⁹mTc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}kr, ²⁰¹TI, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

In various embodiments, a system is provided that include one or more delivery devices for delivering first and second compositions to a subject.

In some embodiments, a delivery device is provided that includes (a) a first reservoir that contains a first fluid composition that comprises an iodinated polyamino compound as described above and (b) a second reservoir that contains a second fluid composition that comprises a reactive multi-arm polymer as described above.

In some embodiments, a delivery device is provided that includes (a) a first reservoir that contains a first fluid composition that comprises an iodinated polyamino compound as described above and a reactive multi-arm polymer as described above, wherein the first fluid composition is buffered to an acidic pH to inhibit covalent crosslinking, such as the prepared fluid composition previously described and (b) a second reservoir that contains a second fluid composition, such as the fluid accelerant composition described above.

In some embodiments, a delivery device is provided that includes (a) a first reservoir that contains a first fluid composition that comprises an iodinated polyamino compound as described above and (b) a second reservoir that contains a second fluid composition that comprises an anionic polymer as described above.

In some embodiments, a delivery device is provided that includes (a) a first reservoir that contains a first fluid composition that comprises an iodinated polyamino compound as described above and an anionic polymer as described above, the first fluid composition being buffered to either an acidic or a basic pH to inhibit ionic crosslinking, such as the prepared fluid composition previously described and (b) a second reservoir that contains a second fluid composition, such as the fluid accelerant composition that is buffered to a basic or acidic pH, as described above.

In each of the above cases, during operation, the first composition and second composition are dispensed from the first and second reservoirs and combined, whereupon the iodinated polyamino compound and the reactive multi-arm polymer and crosslink with one another to form a hydrogel.

Regardless of the first and second compositions selected, in particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

In some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging techniques is an X-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments. As another example, although iodine groups are described above, other radiopaque halogen groups including bromine may be substituted. The invention's scope is, of course, defined in the language in which the appended claims are expressed.

1. A system for forming a hydrogel that comprises an iodinated polyamino compound having a plurality of primary amine groups, all of which are identical, and a polymer that that forms crosslinks with the iodinated polyamino compound.

2. The system of claim 1, wherein the iodinated polyamino compound comprises a polyamino moiety comprising the plurality of primary amine groups that is linked to an iodinated moiety by an amide group.

3. The system of claim 2, wherein the plurality of primary amine groups are alkylamino groups.

4. The system of claim 3, wherein the alkylamino groups are of the formula —(CH₂)ₓ—NH₂, where x is 1, 2 3, 4, 5 or 6.

5. The system of claim 3, wherein all of the alkylamino groups are attached to a single carbon atom.

6. The system of claim 2, wherein the polyamino moiety is a —(CH₂)ₓC((CH₂)ₓ—NH₂)₃ moiety, where x is 1, 2 3, 4, 5 or 6.

7. The system of claim 2, wherein the iodinated moiety comprises an iodinated aromatic group.

8. The system of claim 2, wherein the iodinated moiety comprises a monocyclic or multicyclic aromatic moiety that is substituted with one or more iodine groups and one or more hydroxyl-containing groups.

9. The system of claim 1, wherein the polymer that that forms crosslinks with the iodinated polyamino compound is reactive multi-arm polymer that comprises a plurality of hydrophilic polymer arms having reactive end groups that covalently crosslink with the plurality of primary amine groups of the iodinated polyamino compound.

10. The system of claim 9, wherein the plurality of hydrophilic polymer arms comprise one or more hydrophilic monomers selected from ethylene oxide, N-vinyl pyrrolidone, 2-alkyl-2-oxazoline, hydroxyethyl acrylate, hydroxyethyl methacrylate, PEG methyl ether acrylate or PEG methyl ether methacrylate, or PNIPAAM.

11. The system of claim 9, wherein the reactive end groups are linked to the plurality of hydrophilic polymer arms by a hydrolysable ester and/or wherein the reactive end groups are electrophilic groups.

12. The system of claim 11, wherein the electrophilic groups are selected from imidazole esters, imidazole carboxylates, benzotriazole esters, or imide esters.

13. The system of claim 1, wherein the polymer that forms crosslinks with the iodinated polyamino compound is an anionic polymer that comprises a plurality of anionic groups that can ionically crosslink with the plurality of primary amine groups of the iodinated polyamino compound.

14. The system of claim 13, wherein the anionic polymer that comprises a plurality of carboxyl groups.

15. The system of claim 1, wherein the system comprises a first precursor composition that comprises the iodinated polyamino compound, a second precursor composition that comprises the polymer that forms crosslinks with the iodinated polyamino compound, and an optional accelerant composition.

16. The system of claim 15, wherein the first precursor composition is provided in a syringe barrel, the second precursor composition is provided in a vial, and the accelerant composition is provided in a syringe barrel.

17. The system of claim 1, further comprising a delivery device.

18. A medical hydrogel formed by crosslinking the iodinated polyamino compound and the polymer that forms crosslinks with the iodinated polyamino compound of the system of claim 1.

19. A method of treatment comprising administering to a subject a mixture that comprises an iodinated polyamino compound having a plurality of primary amine groups, all of which are identical, and a polymer that forms crosslinks with the iodinated polyamino compound under conditions such that the iodinated polyamino compound and the polymer crosslink after administration.

20. A method of forming an iodinated polyamino compound comprising (a) forming a partially protected polyamino compound by protecting all but one primary amine group of a polyamino precursor compound comprising three or more primary amine groups, (b) forming an amide linkage between a remaining unprotected primary amine group of the partially protected polyamino compound and a carboxyl group of a carboxylated iodinated precursor compound, and (c) deprotecting primary groups of the product of step (b).
